(19) United States
(12) Patent Application Publication
(10) Pub. No.: US 2018/0055432 A1
(43) Pub. Date: Mar. 1, 2018

(54) BLOOD GLUCOSE MONITORING DEVICE

(71) Applicant: CHUN-YU CHEN, TAIPEI (TW)

(72) Inventor: CHUN-YU CHEN, TAIPEI (TW)

(21) Appl. No.: 15/654,723

(22) Filed: Jul. 20, 2017

(30) Foreign Application Priority Data
Aug. 28, 2016 (TW) 105213038

(51) Int. Cl. A61B 5/15; A61B 5/145; A61B 5/00; G06F 19/00; G01N 27/327

(52) U.S. Cl. CPC A61B 5/150793; A61B 5/150847; A61B 5/150022; G01N 27/3273; A61B 5/150358; A61B 5/7435; G06F 19/322; A61B 5/14532

(57) ABSTRACT

A blood glucose monitoring device includes a container, blood glucose test strips and a glucometer. The container includes a body and a cap. The cap or the body has thereon a rewritable signal component preconfigured with a test strip information. The test strip information includes traceability of the blood glucose test strips. The rewritable signal component sends by wireless communication technology an electronic signal carrying the test strip information. The glucometer includes a casing, processing module built-in with a comparison data, control panel, test slot, and signal processing component. The signal processing component receives the electronic signal of the container by the wireless communication technology. The processing module determines whether the test strip information matches the comparison data and creates a container-opened message when the determination is affirmative, thereby allowing the blood glucose test strips to be for use by the glucometer.

### BACKGROUND OF THE INVENTION

### 1. Technical Field

The present invention relates to blood glucose monitoring devices and, more particularly, to a blood glucose monitoring device which ensures that blood glucose test strips are used up before their expiry dates.

### Description of the Prior Art

Conventional blood glucose test strips have unique lot numbers and parameters, respectively. A conventional blood glucose monitoring device reads their lot numbers and adjusts their parameters, whereas a user has to compare their codes for correctness; the aforesaid requirements are hardly user friendly. Furthermore, a conventional container for blood glucose test strips consists of a can body and a cap. A user puts the test strips in the can body and then closes the can body with the cap so that the closed space in the can body not only contains the test strips but also keeps them dry and thus functioning. In addition to the need to stay dry, the test strips need to be used before expiry dates in order for the blood glucose tests to be accurate. However, using up all the test strips in one's possession before an expiry date is seldom easy to a typical user who requires long-term blood glucose monitoring; it is because once a normal blood glucose test result is yielded, users' complacency is often accompanied by reluctance to take a blood glucose test daily. The aforesaid failure to use up all the test strips in one's possession before an expiry date contributes to inaccuracy in the blood glucose tests, causes a waste of test strips, and compromises the users' voluntary health management.

### BRIEF SUMMARY OF THE INVENTION

It is an objective of the present invention to provide a blood glucose monitoring device which sends an alert or reminder message upon determination that test strips have expired or their lot numbers are rejected by a glucometer.

The blood glucose monitoring device of the present invention uses a rewritable signal component of a container to sense a glucometer and thereby determines whether the glucometer matches blood glucose test strips, thus allowing the glucometer to create a container-opened message which serves as a comparison basis of a check in the future. In brief, according to the present invention, before their use, the blood glucose test strips must sense the container and the glucometer in order to ensure normal and effective use of the blood glucose test strips. Hence, the blood glucose monitoring device of the present invention effectively overcomes drawbacks of the prior art (test strips which have expired are still in use, and test strips do not match the glucometer) and uses an alert message to preclude imprecise blood glucose level test results which might otherwise occur because the test strips have expired or because the container does not sense the glucometer before being opened and used while users are uninformed.

### BRIEF DESCRIPTION OF THE DRAWINGS

FIG. 1 is a schematic perspective view of a container of the present invention;

FIG. 2 is a schematic perspective view of a glucometer of the present invention;

FIG. 3 is a schematic perspective view of a blood glucose test strip of the present invention;

FIG. 4 is a schematic view of an aspect of operation of the present invention; and

FIGS. 5, 6, 7 are schematic views of another aspect of operation of the present invention.

### DETAILED DESCRIPTION OF THE EMBODIMENT OF THE INVENTION

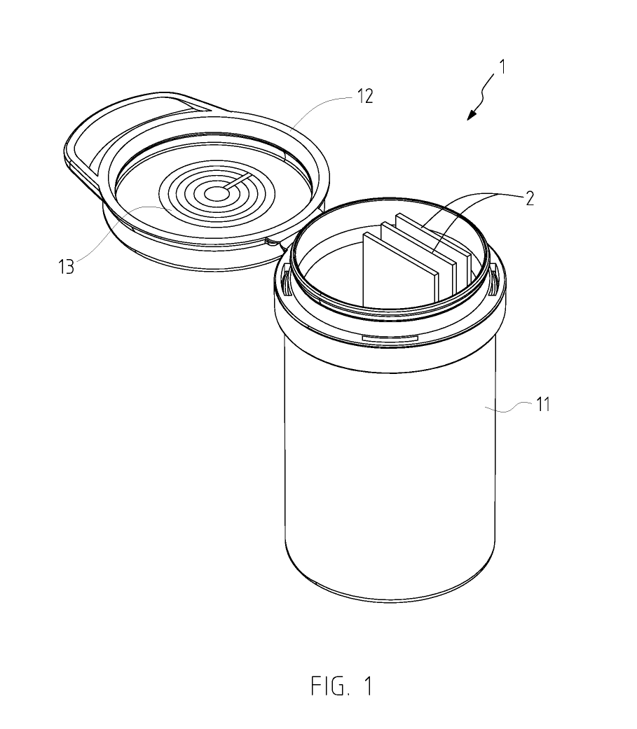

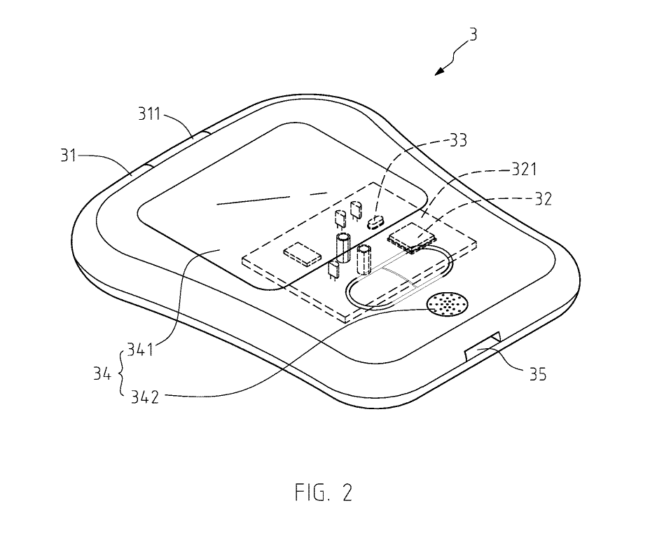

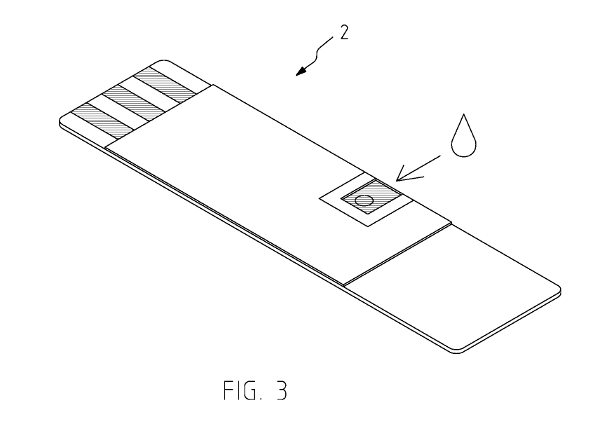

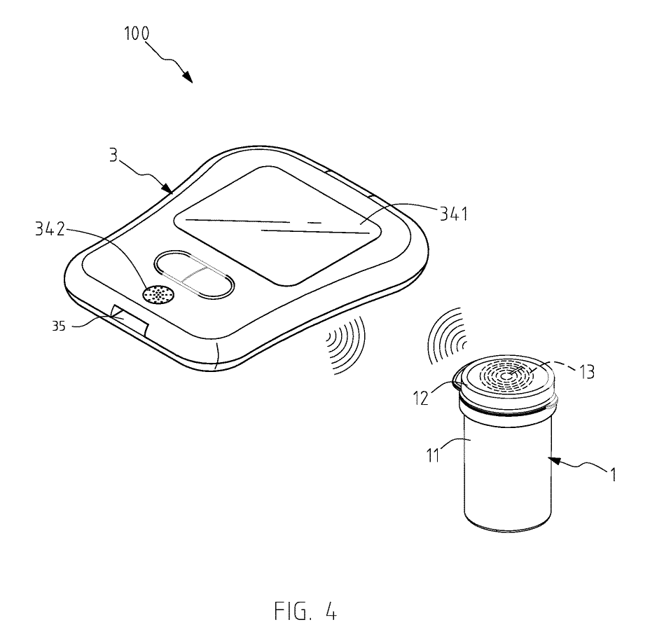

Referring to FIGS. 1-3, in a preferred embodiment of the present invention, a blood glucose monitoring device 100 adopts a commercially-available conventional glucose oxidase electrode measurement technique which involves measuring a current generated as a result of the reaction between blood glucose and glucose oxidase coated on a test strip so as to measure blood glucose levels. The blood glucose monitoring device 100 of the present invention comprises a container 1, 50 blood glucose test strips 2 and a glucometer 3 (as shown in FIG. 4). The container 1 comprises a body 11 and a cap 12 which covers the body 11 tightly. The blood glucose test strips 2 are stored inside the body 11. In this preferred embodiment, the blood glucose test strips 2 expire in three months; hence, blood glucose tests conducted with the blood glucose test strips 2 will be accurate, only if conducted before the expiry date of the blood glucose test strips 2. Designed to suit the glucose oxidase electrode measurement technique, the blood glucose test strips 2 each comprise electrically conductive bands and a reaction layer (as shown in FIG. 3). At the moment when each batch of blood glucose test strips is delivered, impedance level varies slightly among the blood glucose test strips. Therefore, the glucometer must read standard parameters of the blood glucose test strips and adjust the standard parameters in accordance with the variation of the impedance level of the blood glucose test strips with a view to ensuring the accuracy in measuring and interpreting the blood glucose levels. In brief, each glucometer 3 in operation must adjust the standard parameters in accordance with the variation of the impedance level of the blood glucose test strips 2 in order to determine the blood glucose level correctly.

A rewritable signal component 13 (shown in FIG. 1) is disposed on the inner side of the cap 12. The rewritable signal component 13 has an antenna and a chip. In this preferred embodiment, the rewritable signal component 13 effectuates non-contact data transmission by near-field communication (NFC). The rewritable signal component 13 is preconfigured with a test strip information which includes traceability of the blood glucose test strips. The traceability comprises lot numbers, an expiry date, and a production date of the blood glucose test strips. An electronic signal carrying the test strip information is sent by near-field communication technology. In another preferred embodiment, the rewritable signal component 13 sends the electronic signal by wireless radio-frequency identification technology.

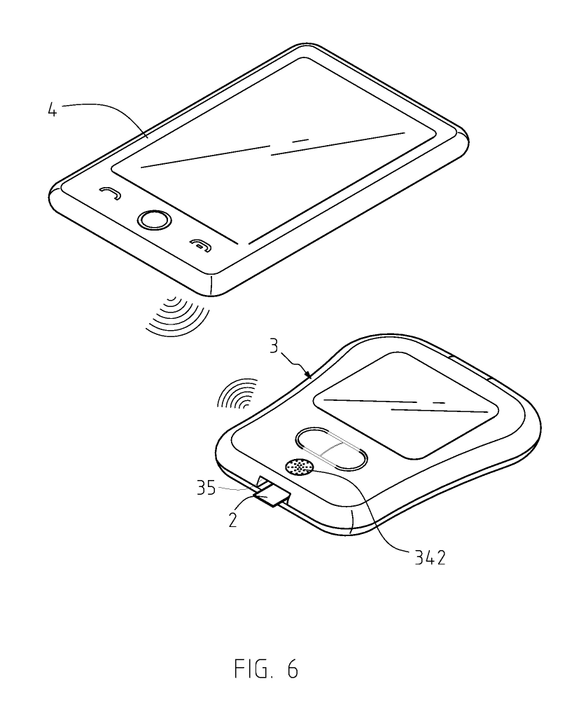

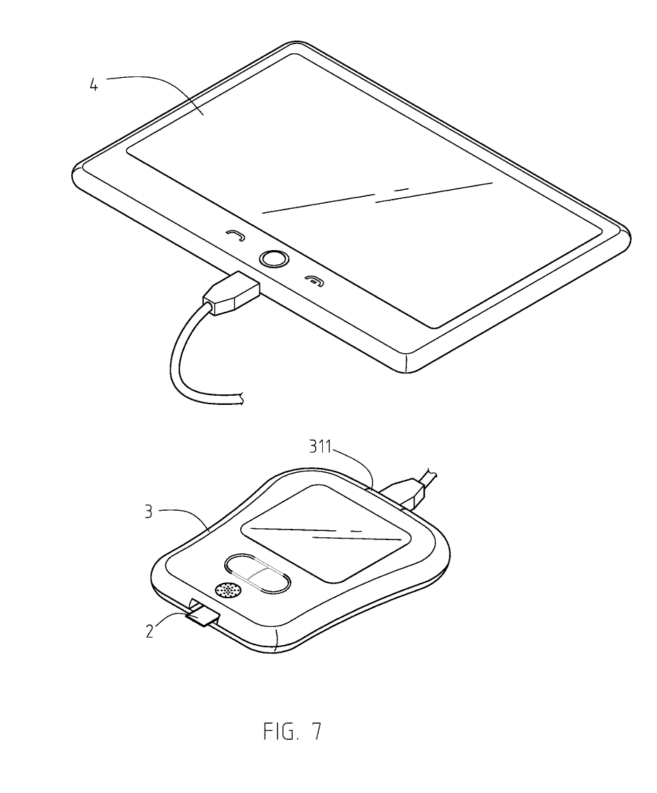

The glucometer 3 comprises a casing 31, a processing module 32 disposed in the casing 31, a control panel 34, a test slot 35 for holding each one of the blood glucose test strips 3, and a signal processing component 33. The control panel 34, the test slot 35 and the signal processing component 33 are electrically connected to the processing module 32. The control panel 34 comprises a circuit board 321. Memory and a processor, both for use in accessing data, a display screen 341 and a speaker component 342 are coupled to the circuit board 321. The signal processing component 33 is disposed in the casing 31. A port 311, such as a USB interface, is disposed on one side of the casing 31, connected to an electronic apparatus 4, such as a tablet (as shown in FIG. 7), and adapted to send resultant blood glucose data to the electronic apparatus 4. In another preferred embodiment, the glucometer 3 sends resultant blood glucose data to the electronic apparatus 4, such as a mobile phone (as shown in FIG. 6), by wireless transmission technology, for example, near-field communication technology. Memory of the processing module 32 is built-in with comparison data which includes traceability, impedance level and correction parameters of test strips.

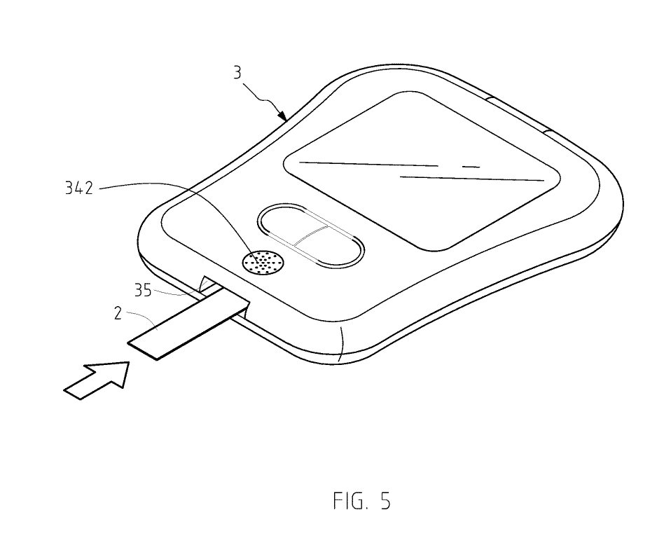

Referring to FIG. 4, the blood glucose monitoring device 100 of the present invention operates in a manner described below. The rewritable signal component 13 of the cap 12 of the container 1 which has not yet been opened approaches the glucometer 3. By wireless communication technology, such as near-field communication, the signal processing component 33 in the glucometer 3 receives the electronic signal sent from the rewritable signal component 13 and sends the electronic signal to the processing module 32 so that the processing module 32 determines whether the test strip information carried by the electronic signal matches the comparison data of the processing module 32. If the determination is negative, either the processing module 32 sends an alert message to be displayed on the display screen 341 by text so as to show that the lot numbers of the test strips do not match the comparison data, or the speaker component 342 generates a voice or sound. If the test strip information carried by the electronic signal matches the comparison data of the glucometer 3, it means that the blood glucose test strips 2 of the container 1 are applicable to the glucometer 3; hence, the processing module 32 of the glucometer 3 creates a container-opened message to be displayed on the display screen 341. Afterward, a user opens the cap 12 and takes out one of the blood glucose test strips 2 for use in a blood glucose test. After taking up blood, the blood glucose test strip 2 is inserted into the test slot 35 (as shown in FIG. 5). The processing module 32 reads the blood glucose level, and then the display screen 341 displays the read blood glucose level. The container-opened message includes the date on which the rewritable signal component 13 of the container 1 sends the electronic signal to the signal processing component 33 of the glucometer 3.

If an expiry date of the blood glucose test strips 2 has passed and the blood glucose test strips 2 are still in use, the processing module 32 will send an alert message which displays text by voice or on the display screen 341 to remind the user that the blood glucose test strips 2 have expired, and the glucometer 3 will not read blood glucose levels. In brief, the blood glucose test strips 2 which have expired are no longer for use by the glucometer 3. In another preferred embodiment, if the container 1 does not sense the glucometer 3 before use, the glucometer 3 will not create the container-opened message, and in consequence the test strip information of the blood glucose test strips 2 in the container 1 cannot be created, preventing the blood glucose test strips 2 in the container 1 which has not been opened and thus confirmed from being for use by the glucometer 3 either.

To prevent the user from not using the container 1 to sense the glucometer 3 before opening the container 1 according to the present invention and thus from complaining about an imprecise test, the present invention is characterized in that the container-opened message created by the glucometer 3 sensed by the container 1 is sent by the wireless communication technology and then written to the rewritable signal component 13 of the container 1. The glucometer 3 or the container 1 sends by the wireless communication technology the comparison data, electronic signal or container-opened message to an electronic apparatus 4, such as a mobile phone (as shown in FIG. 6). Afterward, an application in the electronic apparatus 4 determines whether the blood glucose test strips 2 have expired or whether the glucometer 3 has created the container-opened message, so as to inform the user that the test is imprecise because of improper use, thereby resolving the complaint effectively and quickly.

According to the present invention, the blood glucose monitoring device 100 operates in a manner as follows: the rewritable signal component 13 of the container 1 senses the glucometer 3 to determine whether the glucometer 3 matches the blood glucose test strips 2 and enable the glucometer 3 to create the container-opened message which serves as a comparison basis of a check in the future. In brief, according to the present invention, before their use, the blood glucose test strips 2 must sense the container 1 and the glucometer 3 in order to ensure normal and effective use of the blood glucose test strips 2. Hence, the blood glucose monitoring device 100 of the present invention effectively overcomes drawbacks of the prior art (test strips which have expired are still in use, and test strips do not match the glucometer 3) and uses an alert message to preclude imprecise blood glucose level test results which might otherwise occur because the test strips have expired or because the container 1 does not sense the glucometer 3 before being opened and used while users are uninformed.

What is claimed is:

1. A blood glucose monitoring device, comprising:
a container comprising a body and a cap, the body receiving therein blood glucose test strips, the cap or the body having thereon a rewritable signal component preconfigured with a test strip information, the test strip information including traceability of the blood glucose test strips, the rewritable signal component sending by wireless communication technology an electronic signal carrying the test strip information; and
a glucometer comprising a casing, a processing module disposed in the casing, a control panel, at least a test slot for holding each one of the blood glucose test strips, and a signal processing component electrically connected to the processing module, the processing module being built-in with a comparison data, the signal processing component receiving the electronic signal of the container by the wireless communication technology, wherein the processing module determines whether the test strip information matches the comparison data and creates a container-opened message when the determination is affirmative, thereby allowing the blood glucose test strips to be tested by the glucometer.

2. The blood glucose monitoring device of claim 1, wherein traceability of the test strip information comprises lot numbers, an expiry date, and a production date of the blood glucose test strips.

3. The blood glucose monitoring device of claim 2, wherein if traceability of the blood glucose test strips does not match the comparison data, the processing module sends an alert message.

4. The blood glucose monitoring device of claim 1, wherein the control panel of the glucometer comprises a display screen which at least displays the test strip information of the container and a blood glucose level read.

5. The blood glucose monitoring device of claim 1, wherein the container-opened message is sent by the wireless communication technology and written to the rewritable signal component of the container.

6. The blood glucose monitoring device of claim 1, wherein the rewritable signal component sends the electronic signal to the signal processing component by one of near-field communication technology and wireless radio-frequency identification technology.

7. The blood glucose monitoring device of claim 1, wherein the container-opened message created by the processing module includes a date on which the rewritable signal component of the container sends the electronic signal to the signal processing component of the glucometer.

8. The blood glucose monitoring device of claim 1, wherein one of the glucometer and the container sends one of the comparison data, the electronic signal and the container-opened message to an electronic apparatus by the wireless communication technology.
